(19) United States
(12) Patent Application Publication
(10) Pub. No.: US 2026/0263735 A1
(43) Pub. Date: Sep. 10, 2026

(54) METHOD, DEVICE, AND SYSTEM FOR STIMULATING BRAIN NETWORK

(71) Applicant: Lee Sol Co.,Ltd., Seoul (KR)

(72) Inventors: Gusung Kwon, Seoul (KR); Seung Woo Lee, Seoul (KR); Hyuk Won, Seoul (KR)

(73) Assignee: Lee Sol Co.,Ltd., Seoul (KR)

(21) Appl. No.: 19/266,344

(22) Filed: Jul. 11, 2025

(30) Foreign Application Priority Data
Mar. 10, 2025 (KR) 10-2025-0030820

(51) Int. Cl. A61M 21/02; A61M 21/00

(52) U.S. Cl. CPC A61M 21/02; A61M 2021/0016; A61M 2021/0022; A61M 2021/0027; A61M 2021/005; A61M 2021/0072; A61M 2205/051; A61M 2205/054; A61M 2205/057; A61M 2205/058; A61M 2205/3327; A61M 2205/50; A61M 2210/06

(57) ABSTRACT

Provided is an apparatus including: a content provider configured to provide preset first content and preset second content to a user in a respective manner; a stimulator configured to deliver a preset stimulus to a brain of the user; and a controller configured to control the stimulator such that the stimulus is delivered to the brain of the user only when the second content is provided to the user, wherein the stimulus is delivered to the brain of the user only when the second content is provided to the user, thereby activating a Salience Network (SN) and inducing a transition of the brain from a Default Mode Network (DMN) to a Central Executive Network (CEN).

### CROSS-REFERENCE TO RELATED APPLICATIONS

This application claims priority to and the benefit thereof under 35 U.S.C. § 119 to Korean Patent Application No. 10-2025-0030820 filed in the Korean Intellectual Property Office on Mar. 10, 2025, the entire contents of which are incorporated herein by reference.

### BACKGROUND

### a Field

This study was supported by the 2023 clinical trial (HI23C0607) on sleep improvement using tACS (transcranial alternating current stimulation) for sleep induction through brainwave modulation, funded by the Korea Health Industry Development Institute (KHIDI).

The present disclosure relates to a brain network stimulating method, apparatus, and system. More specifically, the present disclosure relates to a brain network stimulating method, apparatus, and system that induces a transition from a Default Mode Network (DMN) to a Central Executive Network (CEN) by activating a Salience Network (SN) in the brain through a specific stimulus when preset content is provided.

### b Description of the Related Art

Recent neuroscience research has revealed that abnormal interactions in brain networks associated with various neuropsychiatric disorders and cognitive functions play an important role.

In this context, a triple-network model has been proposed, which is based on the concept that an interaction among the Default Mode Network (DMN), Salience Network (SN), and Central Executive Network (CEN) plays a central role in mental health and cognitive regulation.

There exist interactions and anticorrelations among the aforementioned networks.

That is, the SN plays a role in switching between the DMN and the CEN, and a process in which the DMN is deactivated and the CEN is activated during a performance of specific tasks is important.

The DMN and the CEN have an anticorrelation relationship, such that when the DMN is activated, an activity of the CEN decreases, and conversely, when the CEN is activated, an activity of the DMN is suppressed.

If the SN does not function normally, the balance between the DMN and the CEN is disrupted, which may result in problems such as attention deficit, excessive internal thought (rumination), or executive dysfunction.

### SUMMARY

The present disclosure may solve the aforementioned problems by providing a brain network stimulating method, apparatus, and system that induces a transition from the DMN to the CEN by activating the SN in the brain through a specific stimulus when preset content is provided.

The problems to be solved by the present disclosure are not limited to those mentioned above and may be extended to various matters derived from embodiments described below.

In some embodiments, a brain network stimulating method, apparatus, and system may be provided, which induce a transition from a Default Mode Network (DMN) to a Central Executive Network (CEN) by activating a Salience Network (SN) of the brain through a specific stimulus when preset content is provided.

Some embodiments may provide an effect of promoting a functional transition of the brain network. By distinguishing between preset first content and second content and providing them selectively, it becomes possible to control the delivery of a preset stimulus to the user's brain only under specific conditions. The stimulus activates the SN of the brain, and the activated SN induces a transition from the DMN to the CEN, thereby enhancing the user's attention and cognitive functions.

In addition, some embodiments may provide an effect of improving working memory and cognitive flexibility. A specific stimulus is delivered to a Dorsolateral Prefrontal Cortex (DLPFC) to activate the SN, thereby enhancing the process in which the DMN is suppressed and the CEN is activated. As a result, the user's working memory and cognitive flexibility are improved, allowing for more efficient information storage and adaptation to new information.

Some embodiments may also provide an effect of enhancing attention control and inhibition control. By promoting the SN activation and the transition to the CEN, the user’s functions of attention control and inhibition control are strengthened. This allows the user to focus on specific information even in a distracting environment and improves the ability to suppress unnecessary actions, thereby enabling effective response in situations requiring high concentration.

Furthermore, some embodiments may provide an effect of activating neural networks through multi-sensory stimulation. The present disclosure may apply multi-modal stimulation using tACS, tDCS (transcranial direct current stimulation), tMS (transcranial magnetic stimulation), TUS (transcranial ultrasound stimulation), tPBM (transcranial photobiomodulation), and sensory stimulation. When such various stimuli are applied in combination, they can produce a higher effect than a single-stimulus method, and allow for neural network activation through multiple senses such as vision, hearing, and touch.

Additionally, some embodiments may provide an effect of CEN activation by using high-frequency gamma oscillation. In order to activate the SN, synchronized oscillation (entrainment) is applied using gamma oscillation of 30 Hz or higher, thereby enhancing user attention and promoting CEN activation. This gamma oscillation plays an important role in learning and cognitive function enhancement, and is designed to be entrained only when specific mission content is provided, enabling more precise control.

Moreover, some embodiments may provide an effect of blocking unnecessary information and optimizing brain function. By designing the user's brain to distinguish between content with low relevance (e.g., random sound and random image) and content with high relevance (e.g., music, environmental sound, voice, etc.), meaningless stimuli (e.g., noise) may be used to activate the DMN, and meaningful stimuli may be used to induce CEN activation. Also, by modulating neural responses to unnecessary information using 8 to 13 Hz alpha (α) waves or 4 to 7 Hz theta (θ) waves, selective responses to information requiring concentration may be induced.

In addition, some embodiments may provide an effect of improving neural network imbalance in patients with mental disorders. Some embodiments may be used to treat various mental disorders by activating the SN and training the transition between the DMN and the CEN. For example, in patients with depression, excessive activation of the DMN and functional degradation of the CEN may be alleviated; in patients with anxiety disorders, the overactivation of the SN and imbalance between the DMN and CEN may be improved. Furthermore, in patients with schizophrenia, functional degradation of the CEN may be mitigated, and in patients with ADHD, an attention deficit problem may be addressed through CEN activation.

Some embodiments may also provide an effect of improving learning and physical performance. By linking specific mission content to learning sounds (English words and idioms) or designating key learning content in lecture content as a specific region, memory and comprehension of the learner may be enhanced. Furthermore, physical performance may be improved by distinguishing between exercises requiring attention and those that do not, and applying specific stimuli accordingly. This can be applied not only to learning environments but also to sports and rehabilitation therapy.

The effects provided in the present disclosure are not limited to those described above and may be extended to various matters derived from the embodiments of the invention described below.

According to an aspect of an embodiment, an apparatus according to an embodiment may include: a content provider configured to provide preset first content and preset second content to a user in a respective manner; a stimulator configured to deliver a preset stimulus to a brain of the user; and a controller configured to control the stimulator such that the stimulus is delivered to the brain of the user only when the second content is provided to the user, wherein the stimulus is delivered to the brain of the user only when the second content is provided to the user, thereby activating a Salience Network (SN) and inducing a transition of the brain from a Default Mode Network (DMN) to a Central Executive Network (CEN).

In some embodiments, the stimulus is delivered to a Dorsolateral Prefrontal Cortex (DLPFC) region to activate the SN.

In some embodiments, when the SN is activated and the brain transitions from the DMN to the CEN, at least one function among working memory, cognitive flexibility, executive function, decision making and problem-solving, attention control, and inhibition control is performed.

In some embodiments, the stimulus is configured to activate the SN by entraining synchronized oscillations in a plurality of regions of the brain.

In some embodiments, the entrained synchronized oscillations comprise gamma oscillations of 30 Hz or higher.

In some embodiments, when the first content is provided to the user, the stimulator is further configured to deliver a preset noise stimulus to the brain of the user, wherein the noise stimulus is a stimulus entraining synchronized oscillations associated with deactivation of the SN, and wherein the synchronized oscillations entrained by the noise stimulus comprise alpha waves (α) of 8 to 13 Hz or theta waves (θ) of 4 to 7 Hz.

In some embodiments, the stimulus comprises at least one of: transcranial alternating current stimulation (tACS), transcranial direct current stimulation (tDCS), transcranial magnetic stimulation (tMS), transcranial ultrasound stimulation (TUS), transcranial photobiomodulation (tPBM), and sensory stimulation, and wherein the sensory stimulation comprises at least one of: visual stimulation, auditory stimulation, tactile stimulation, olfactory stimulation, and gustatory stimulation.

In some embodiments, the stimulus for the entrainment comprises multi-stimulation in which at least two or more among the tACS, tDCS, tMS, TUS, tPBM, and sensory stimulation are simultaneously applied.

In some embodiments, the first content is noise content having a low degree of relevance to the user, and wherein the second content is mission content having a high degree of relevance to the user.

In some embodiments, the first content comprises sound information including at least a random sound, wherein the second content comprises sound information including at least one of music related to the user, environment sound related to the user, and voice related to the user, and wherein only when the second content is provided, the gamma oscillation is entrained, and the gamma oscillation entrainment activates the SN to induce a transition from the DMN to the CEN.

In some embodiments, the second content comprises learning sound information including at least one of preset English words and preset idioms that the user intends to learn.

In some embodiments, the first content comprises image information including at least a random image unrelated to the user, wherein the second content comprises image information including at least an image related to the user, and wherein only when the second content is provided, gamma oscillation is entrained, and the gamma oscillation entrainment activates the SN to induce a transition from the DMN to the CEN.

In some embodiments, the first content comprises both sound information including at least a random sound, and image information including at least a random image unrelated to the user, wherein the second content comprises both sound information including at least one of music related to the user, environment sound related to the user, and voice related to the user, and image information including at least an image related to the user, and wherein only when the second content is provided, gamma oscillation is entrained, and the gamma oscillation entrainment activates the SN to induce a transition from the DMN to the CEN.

In some embodiments, the first content is lecture content information that the user is learning, wherein the second content is specific lecture region information including preset important learning content among the lecture content information, and wherein only when the second content is provided, gamma oscillation is entrained, and the gamma oscillation entrainment activates the SN to induce a transition from the DMN to the CEN.

In some embodiments, the first content is random information guiding the user's movement performed without concentration by the user, and wherein the second content is information guiding the user's movement that requires concentration by the user, and wherein only when the second content is provided, gamma oscillation is entrained, and the gamma oscillation entrainment activates the SN to induce a transition from the DMN to the CEN.

In some embodiments, when the user is a patient with depression, the stimulus activates the SN, and based on the activation of the SN, the transition process between the DMN and the CEN is trained, thereby solving the problem of excessive activation of the DMN and functional degradation of the CEN in the patient with depression.

In some embodiments, when the user is a patient with an anxiety disorder, the stimulus activates the SN, and based on the activation of the SN, the transition process between the DMN and the CEN is trained, thereby solving the problem of excessive activation of the SN and imbalance between the DMN and the CEN in the patient with an anxiety disorder.

In some embodiments, when the user is a patient with schizophrenia, the stimulus activates the SN, and based on the activation of the SN, the transition process between the DMN and the CEN is trained, thereby solving the problems of functional degradation of the CEN and abnormality of the SN in the patient with schizophrenia.

In some embodiments, when the user is a patient with ADHD, the stimulus activates the SN, and based on the activation of the SN, the transition process between the DMN and the CEN is trained, thereby solving the problem of functional degradation of the CEN in the patient with ADHD.

According to an aspect of an embodiment, a brain network monitoring method according to an embodiment may include: providing preset first content and preset second content to a user in a respective manner; delivering a preset stimulus to a brain of the user; controlling so that the stimulus is delivered to the brain of the user only when the second content is provided to the user; and monitoring whether the brain transitions from a Default Mode Network (DMN) to a Central Executive Network (CEN) by activating a Salience Network (SN) through the stimulus.

### BRIEF DESCRIPTION OF THE DRAWINGS

The above and other aspects, features, and advantages of certain embodiments of the present disclosure will become apparent from the following description taken in conjunction with the accompanying drawings, in which:

FIGS. 1A and 1B are diagrams for illustrating a large-scale brain network.

FIG. 2 is a diagram for illustrating a triple-network model, which is a brain network model that facilitates switching between the DMN and the CEN by the SN.

FIGS. 3A and 3B are diagrams for illustrating the triple-network model and mental disorders.

FIG. 4 is a diagram illustrating an example of network normalization through auditory stimulation.

FIG. 5 is a diagram illustrating an example of network normalization through visual stimulation.

FIG. 6 is a diagram illustrating an example of network normalization through sensory-motor stimulation.

FIG. 7 is a diagram illustrating an example of network normalization through visual stimulation, sound, and tACS stimulation based on a concept related to English learning.

FIG. 8 is a diagram illustrating an example of network normalization through visual stimulation, sound, and tACS stimulation based on a concept related to online lecture learning.

FIG. 9 is a diagram illustrating an embodiment of using visual stimulation, sound, and tACS based on a concept of online lecture learning according to an embodiment.

FIG. 10 is a diagram illustrating an example of implementing online lecture learning according to an embodiment.

FIG. 11 is a diagram illustrating an embodiment applied to online lectures.

FIG. 12 is a diagram illustrating an example of a brain network stimulating apparatus and system according to an embodiment.

FIG. 13 is a diagram showing a dFNC variability analysis graph and a comparison chart of connectivity between networks.

FIG. 14 is a diagram showing a dFNC state analysis chart.

### DETAILED DESCRIPTION OF THE INVENTION

Hereinafter, specific details for implementing the disclosure will be described in detail with reference to the accompanying drawings. However, in the following description, detailed descriptions of well-known functions or configurations that may unnecessarily obscure the gist of the disclosure will be omitted.

In the accompanying drawings, the same or corresponding components are given the same reference numerals. In addition, in the following description of the disclosure, redundant descriptions of the same or corresponding components may be omitted. However, the omission of the description regarding certain components does not intend to imply that such components are not included in the disclosure.

Advantages and features of the disclosure, and methods for achieving them, will become apparent with reference to the embodiments described below together with the accompanying drawings. However, the disclosure is not limited to the embodiments set forth below but may be implemented in various different forms, and these embodiments are provided merely to ensure completeness of the disclosure and to fully convey the scope of the invention to those skilled in the art.

The terms used in the disclosure will be briefly explained, and the disclosure will be specifically described. The terms used in the disclosure have been selected as general terms currently widely used, considering the functions in the disclosure, however, they may vary depending on the intention of a person skilled in the art, judicial precedents, or the emergence of new technologies. Also, in specific cases, there may be terms arbitrarily selected by the applicant, in which case the meaning will be clearly described in the relevant part of the disclosure. Therefore, the terms used in the disclosure should be defined based not merely on their names but based on their meanings and the overall content of the disclosure.

In the disclosure, singular expressions shall include plural expressions unless clearly specified as singular in context. Likewise, plural expressions shall include singular expressions unless clearly specified as plural in context. When a part of the disclosure states that a component "includes" another component, it is to be understood that, unless expressly stated otherwise, the component does not exclude other components and may further include additional components.

When describing the embodiments of the disclosure, detailed descriptions of well-known configurations or functions may be omitted if they are deemed to obscure the essential features of the disclosure. Also, in the drawings, parts irrelevant to the description of the disclosure are omitted, and similar parts are denoted by similar reference numerals.

In the disclosure, when a component is described as being "connected to," "coupled to," or "joined to" another component, it may include not only a direct connection but also an indirect connection through another component in between. Also, when a component is said to "include(comprise)" or "have" another component, it means that other components may be further included unless specifically stated otherwise.

In the disclosure, terms such as "first," "second," and so on are merely used to distinguish one component from another and do not limit the order or importance of the components unless otherwise specified. Therefore, a first component in one embodiment may be referred to as a second component in another embodiment, and likewise, a second component in one embodiment may be referred to as a first component in another embodiment.

In the disclosure, distinct components are described to clearly explain their respective features and do not necessarily imply that they are separate. That is, multiple components may be integrated into a single hardware or software unit, or one component may be distributed and implemented across multiple hardware or software units. Accordingly, unless stated otherwise, such integrated or distributed embodiments are also within the scope of the disclosure.

In the disclosure, the term "network" may include both wired and wireless networks. In this case, the network may refer to a communication network through which data exchange is performed between devices and systems or between devices, and is not limited to a specific type of network.

The embodiments described in the disclosure may be entirely hardware-based, partly hardware-based and partly software-based, or entirely software-based. The terms "unit," "device," or "system" used in the disclosure refer to computer-related entities, which may be hardware, a combination of hardware and software, or software. For example, in the disclosure, a unit, a module, a device, or a system may be a running process, processor, object, executable file, thread of execution, program, and/or computer, but is not limited thereto. For example, both an application running on a computer and the computer itself may correspond to a unit, a module, a device, or a system in the disclosure.

Also, in the disclosure, the term "device" may refer not only to mobile devices such as smartphone, tablet PC, wearable device, and head mounted display (HMD), but also to fixed devices such as PC or home appliance with display function. As an example, the device may be a cluster inside a vehicle or an IoT (Internet of Things) device. That is, in the disclosure, a device may refer to any device on which an application can operate and is not limited to a specific type. For convenience of explanation, a device on which an application operates is referred to as a device.

In the disclosure, the communication method of the network is not limited, and connections between respective components may not be based on the same network type. The network may include not only communication methods utilizing communication networks (e.g., mobile communication networks, wired internet, wireless internet, broadcasting networks, satellite networks), but also short-range wireless communication between devices. For example, the network may include all communication methods by which objects can network, and is not limited to wired communication, wireless communication, 3G, 4G, 5G, or other methods. For example, the network may refer to a communication network by one or more communication methods selected from among LAN (Local Area Network), MAN (Metropolitan Area Network), GSM (Global System for Mobile Network), EDGE (Enhanced Data GSM Environment), HSDPA (High Speed Downlink Packet Access), W-CDMA (Wideband Code Division Multiple Access), CDMA (Code Division Multiple Access), TDMA (Time Division Multiple Access), Bluetooth, Zigbee, Wi-Fi, VoIP (Voice over Internet Protocol), LTE Advanced, IEEE802.16m, WirelessMAN-Advanced, HSPA+, 3GPP LTE (Long Term Evolution), Mobile WiMAX (IEEE 802.16e), UMB (formerly EV-DO Rev. C), Flash-OFDM, iBurst and MBWA (IEEE 802.20) systems, HIPERMAN, BDMA (Beam-Division Multiple Access), Wi-MAX (World Interoperability for Microwave Access), and communication using ultrasonic waves, but is not limited thereto.

The components described in various embodiments do not necessarily mean that they are essential components, and some may be optional components. Therefore, embodiments composed of a subset of the components described in the disclosure are also within the scope of the disclosure. Additionally, embodiments that further include other components in addition to those described in various embodiments are also within the scope of the disclosure.

Before describing specific contents of the present disclosure, a large-scale brain network will be described.

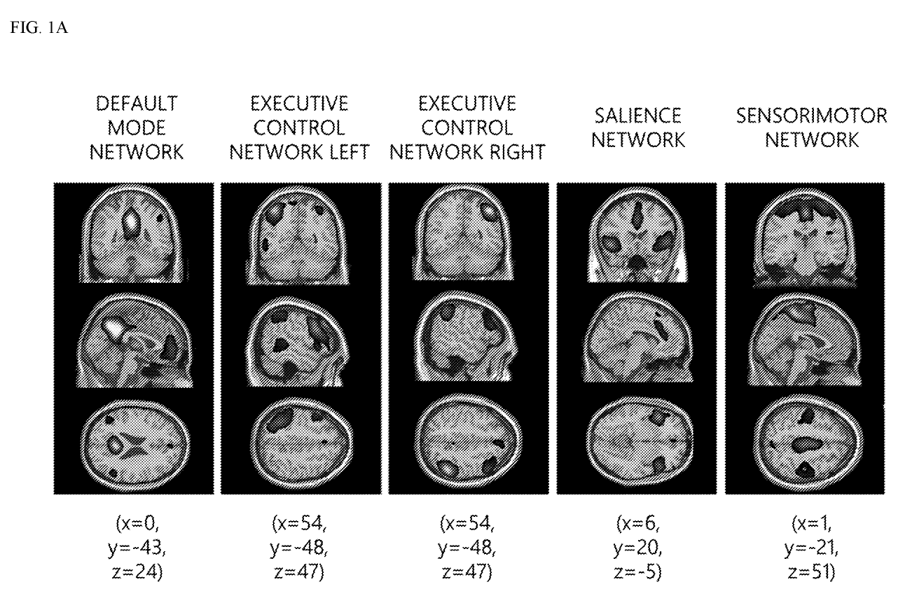

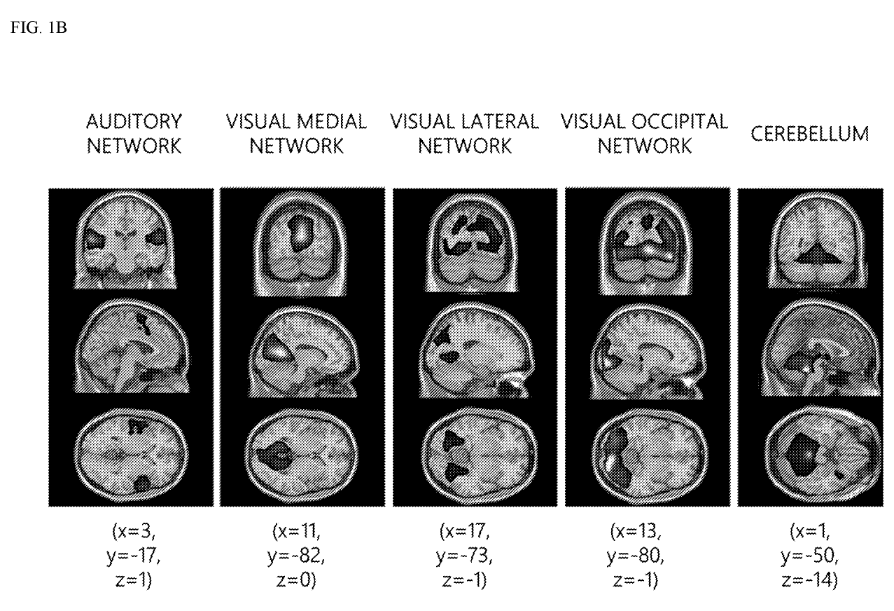

FIGS. 1A and 1B are diagrams for illustrating a large-scale brain network.

Referring to FIGS. 1A and 1B, the large-scale brain network includes a Default Mode Network (DMN), a Salience Network (SN), a Dorsal Attention Network (DAN), a Frontoparietal Network (FPN), a Central Executive Network(CEN), a Sensorimotor Network (SMN), a Visual Network (VN), and an Auditory Network (AN).

First, the DMN is a network activated during a resting state when awake.

The DMN is primarily activated when performing internally directed tasks such as self-referential thinking, self-reflection, memory recall, future planning, and social cognition, rather than processing external stimuli.

Next, the SN is a network responsible for detecting and filtering salient stimuli.

The SN identifies perceptually prominent elements in the external environment or internally important information and is involved in regulating cognitive and behavioral responses.

It functions to regulate switching between the DMN and the CEN and plays an important role in a process of determining whether specific stimuli are cognitively important.

In addition, the DAN is a network that goal-directedly controls spatiotemporal attention.

The DAN is activated in the process of attention control reflecting the user's intention and performs active attention control for specific objects or events.

In addition, the FPN is a network responsible for executive functions and goal-directed cognitive tasks.

The FPN is involved in cognitively demanding tasks such as complex problem-solving, decision-making, working memory, and attention control.

It is also referred to as the CEN and plays a key role in performing high-level cognitive control.

In addition, the SMN is a network that processes somatosensory information and is responsible for motor control.

The SMN integrates various sensory inputs related to motor planning and execution and plays a role in coordinating body movement.

In addition, the VN is a network involved in processing visual information.

The VN is responsible for various visual functions such as detection, recognition, spatial processing of visual stimulation, and interpretation of shape and color of objects.

It is activated in various visual-related regions including the visual cortex.

In addition, the AN is a network that processes auditory information.

The AN plays an important role in detecting and interpreting auditory stimuli, voice recognition, music appreciation, and language processing.

It is activated in regions associated with the auditory cortex and also affects auditory attention control.

The aforementioned large-scale brain networks are closely interconnected and interact with each other during cognitive function execution and behavior regulation processes.

Next, the background of the proposal of the present disclosure will be described.

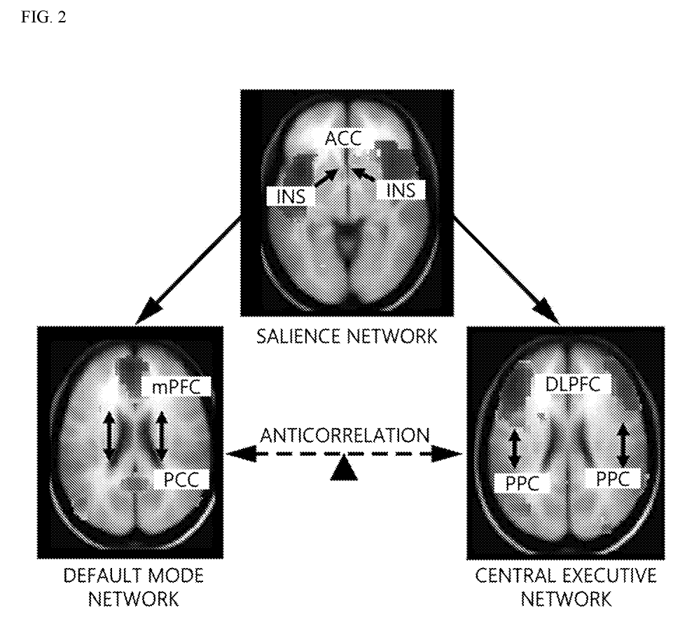

FIG. 2 is a diagram for illustrating a triple-network model, which is a brain network model that facilitates switching between the DMN and the CEN by the SN.

Referring to FIG. 2, recent neuroscience studies have revealed that abnormal interactions among brain networks related to various neuropsychiatric disorders and cognitive functions play an important role.

In this background, a triple-network model has been proposed, which is based on the concept that interactions among the DMN, SN, and CEN play a central role in mental health and cognitive regulation.

As described above, the DMN is a network that regulates activities related to internal thought, self-referential processing, memory retrieval, and self-reflection.

It is mainly activated in the medial prefrontal cortex (mPFC) and the posterior cingulate cortex (PCC).

It is activated when focusing on internal information processing rather than changes in the external environment and tends to be deactivated when attention is directed to the external environment.

In addition, the SN plays a key role in detecting salient stimuli from internal and external environments and regulating switching between the DMN and the CEN based on such information.

Major components include the anterior cingulate cortex (ACC) and the insular cortex (INS), which are involved in emotion regulation, evaluation of the salience of stimuli, and triggering behavioral responses.

When the function of the SN is impaired, it may become difficult to regulate attention, or the balance between specific networks may collapse.

In addition, the CEN is a network that performs higher-order cognitive functions and is responsible for functions such as attention control, working memory, decision-making, and problem solving.

It is primarily activated in the DLPFC and a posterior parietal cortex (PPC).

It plays an important role in the process of adapting to complex cognitive demands imposed externally and strategically thinking to solve them.

Interactions and anticorrelations exist among the aforementioned networks.

That is, the SN plays a role in regulating switching between the DMN and the CEN, and the process in which the DMN is deactivated and the CEN is activated during the performance of a specific task is important.

The DMN and the CEN have an anticorrelation relationship, in which the activity of the CEN decreases when the DMN is activated, and conversely, the activity of the DMN is suppressed when the CEN is activated.

If the SN does not function properly, the balance between the DMN and the CEN may collapse, causing problems such as attention deficit, excessive internal thought (e.g., rumination), or executive dysfunction.

Accordingly, some embodiments may provide a method, apparatus, and system that can provide treatment and management based on the triple-network model.

The triple-network model has been proposed as a model to explain cognitive dysfunction observed in mental disorders and neurocognitive impairments.

For example, in a patient with depression, excessive activation of the DMN may exacerbate rumination, and the function of the CEN is likely to deteriorate.

In addition, in schizophrenia, due to impaired regulatory function of the SN, smooth switching between the DMN and the CEN may not occur, resulting in reduced reality testing function.

Also, in attention-deficit/hyperactivity disorder (ADHD), it may become difficult to regulate attention and executive functions due to impaired function of the CEN and abnormal regulation by the SN.

In addition, in anxiety disorder, excessive activation of the SN may lead to overreaction to external threat signals.

Accordingly, the triple-network model may provide an important framework for understanding various mental disorders and cognitive dysfunctions, and may contribute to the development of treatments by regulating abnormal interactions among such networks.

The relationship between the triple-network model and mental disorders will be described in more detail.

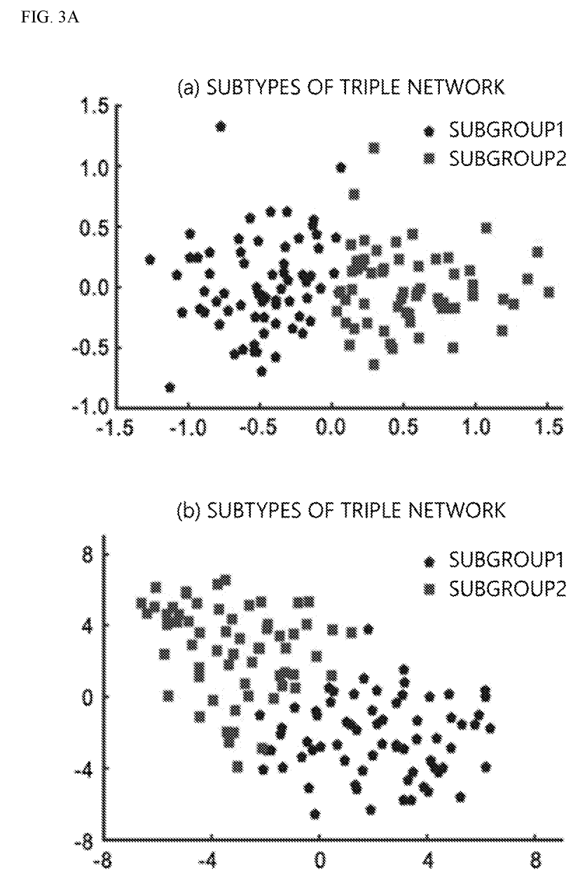

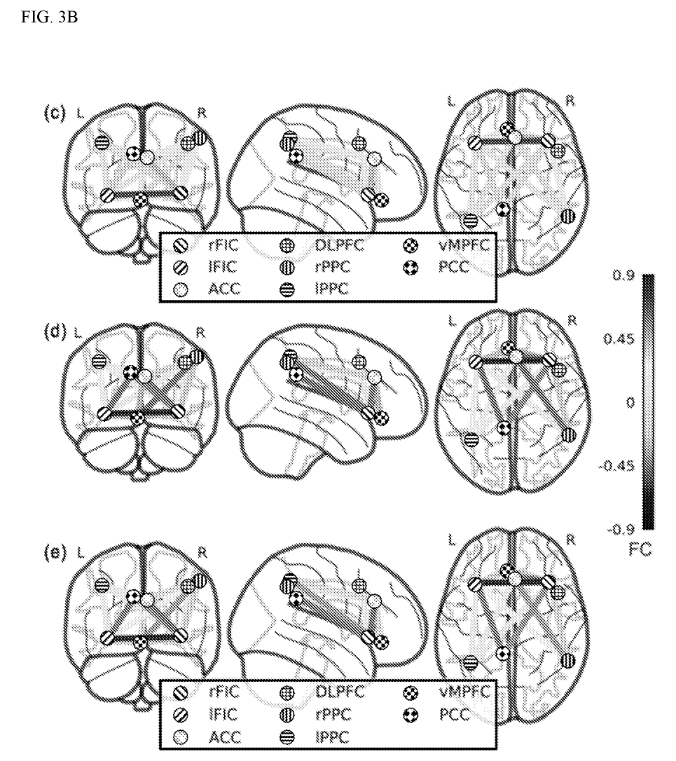

FIGS. 3A and 3B are diagrams for illustrating a triple-network model and mental disorders.

Referring to FIGS. 3A and 3B, recent neuroscience research emphasizes that functional abnormalities in a brain network are closely related to various mental disorders, and that understanding and regulating the functions of such networks is a core challenge in the treatment of mental disorders.

As described above, the triple-network model is based on the concept that interactions among the DMN, the SN, and the CEN play a central role in mental health and cognitive function regulation.

Referring to FIG. 3A, based on the triple-network model, changes in brain networks of patients with mental disorders were analyzed, and graphs (a) and (b) visually illustrate distributions by dividing patients showing specific types of mental disorders (i.e., Subgroup 2) and a healthy control group (i.e., Subgroup 1).

In addition, referring to FIG. 3B, illustrations (c), (d), and (e) show functional connectivity (FC) among various regions within the triple-network, where the strength of connectivity between networks is expressed differently according to color (or grayscale intensity).

Regarding the correlation between the triple-network model and mental disorders, abnormalities in each network may serve as major causes of various mental disorders, and excessive activation or functional decline of a specific network is a common feature observed in patients with mental disorders.

First, in case of depression, excessive activation of the DMN and functional decline of the CEN may occur.

This is related to the fact that patients with depression have a strong tendency toward rumination and continuously recall past negative experiences, thereby facing difficulties in realistic problem solving.

In a normal case, the SN regulates the switching between the DMN and the CEN, but in patients with depression, the SN may fail to induce appropriate switching, resulting in a sustained overactivation of the DMN.

As a result, patients may experience reduced concentration, difficulties in decision-making, and challenges in setting future plans.

In addition, in case of anxiety disorders, excessive activation of the SN and imbalance between the DMN and the CEN may occur.

Patients with anxiety disorders show enhanced hypervigilance, and the SN is excessively activated, displaying a tendency to constantly stay alert to external stimuli.

In a normal case, the SN filters important information so that the CEN may process it appropriately, but patients with anxiety disorders tend to interpret unnecessary information as danger signals due to dysfunction of the SN.

As a result, continuous worrying and fear responses occur, and anxiety may be easily felt even when there is no realistic threat.

In addition, schizophrenia may be characterized by functional decline of the CEN and dysfunction of the SN.

Patients with schizophrenia exhibit a reduced ability to distinguish between reality and illusion, which is associated with the functional decline of the CEN.

Furthermore, as the regulatory function of the SN becomes impaired, external stimuli and internal thoughts may not be appropriately distinguished, resulting in a high likelihood of symptoms such as delusions or auditory hallucinations.

When the SN fails to properly perform its role of regulating the switching between the DMN and the CEN, patients with schizophrenia may exhibit distorted cognitive interpretations of the external world.

Such abnormalities in functional connectivity are reported to be related to structural abnormalities during neural development or to imbalances in neurotransmitters.

In addition, attention deficit hyperactivity disorder (ADHD) may be characterized by functional decline of the CEN.

Patients with ADHD experience difficulty in attention control, which is due to the impaired operation of executive function caused by decline of the CEN.

In addition, problems in inhibition control and working memory may occur, resulting in difficulty in maintaining sustained attention in learning or daily life, and a tendency to exhibit impulsive behavior.

In case of a patient with ADHD, the SN may fail to properly perform the role of filtering unimportant information, thereby potentially preventing the DMN from being properly deactivated, and as a result, excessive internal thought and reduced concentration may occur.

Therefore, research based on the triple-network model provides important implications for the treatment of mental disorders.

Maintaining balance among the DMN, SN, and CEN is essential for mental health, and hyperactivation or functional decline of a specific network may become a cause of mental disorders.

In each mental disorder, patterns of functional connectivity (e.g., fMRI-based analysis) for specific networks appear differently, and these may be utilized to design objective diagnostic assistance and customized treatment strategies.

Some embodiments may provide a method for treating mental disorders by regulating an activity level of a specific network through treatment techniques such as non-invasive neuromodulation.

The triple-network model may provide an important conceptual framework for understanding mental disorders, and abnormalities in functional connectivity among the DMN, the SN, and the CEN may serve as one of the major causes of mental disorders.

In order to treat mental disorders, it is important to restore harmonious function among the triple-networks, and new treatment methods based on this need to be developed. In future research, more sophisticated intervention methods should be studied so that such network-based treatment strategies can make practical contributions to the improvement of mental health.

Hereinafter, a method according to an embodiment will be described in more detail with reference to the drawings.

The present disclosure may provide a brain network stimulating method that adjusts a balance among the DMN, SN, and CEN based on a triple-network model.

Some embodiments may activate the SN through a specific stimulus, thereby inducing smooth switching between the DMN and the CEN, enhancing cognitive function, and improving mental disorders.

Some embodiments may provide a brain network stimulating method that includes providing preset first content and second content to a user in a distinguished manner (or in a respective manner), and delivering a specific stimulus to a brain of the user only when the second content is provided.

At this time, the stimulus activates the SN and induces promotion of switching from the DMN to the CEN.

According to the triple-network model, the SN is a core network that controls the switching between the DMN and the CEN, and the activation of the SN leads to a transition from internally directed thinking to cognitive response to external stimuli.

The stimulus applied in some embodiments may be delivered to the DLPFC so as to effectively activate the SN.

The DLPFC is a core region of the CEN, and by stimulating this region, it plays a role in enhancing executive function and suppressing hyperactivation of the DMN.

When the SN is activated in the above-described manner and switching from the DMN to the CEN is induced, an effect of improving cognitive function of the brain may be expected.

In particular, functions such as working memory, cognitive flexibility, executive function, decision making and problem-solving, attention control, and inhibition control may be enhanced.

When a stimulus is applied to the DLPFC, the CEN is activated, which helps the user to escape from the DMN and maintain concentration.

In addition, the activation of the SN regulates switching between the DMN and the CEN to activate an appropriate network depending on the situation.

That is, through direct stimulation to the DLPFC and activation of the SN, improvement in attention control, enhancement in executive function, and improvement in cognitive flexibility may be achieved.

These functions are essential high-level cognitive abilities in mental health and daily life, and the present disclosure may enhance them in a neuroscientific manner.

In addition, a stimulation method according to an embodiment may be more precisely improved in a manner of activating the SN by inducing entrainment of synchronized oscillations in a plurality of regions of the brain.

According to the triple-network model, the SN is connected with various brain regions, and neural entrainment at a specific frequency is effective for network activation.

Entrainment of synchronized oscillations in the brain refers to a phenomenon in which brainwaves at a specific frequency are harmonized and operate synchronously across multiple brain regions. Such synchronization plays an important role in strengthening neural network connectivity related to cognitive function, emotion regulation, memory, and attention.

The brain generates synchronized rhythms at specific frequencies, and these rhythms contribute to optimizing information exchange between neural circuits. The frequency bands of the brain are classified into five types, each having different functions.

Delta waves (0.5 to 4 Hz) are activated during deep sleep and physical recovery processes and are synchronized in a thalamus and cerebral cortex.

Theta waves (4 to 8 Hz) are related to creativity, memory, and learning, and are mainly synchronized in a hippocampus, frontal lobe, and septal nuclei.

Alpha waves (8 to 14 Hz) occur in relaxed and focused states and are activated in an occipital lobe, parietal lobe, and frontal lobe.

Beta waves (14 to 30 Hz) are related to logical thinking, problem solving, and working memory, and are synchronized in the frontal lobe and somatosensory cortex.

Gamma waves (30 to 100 Hz) regulate high-level cognition and conscious states, and are synchronized in the frontal lobe, parietal lobe, and hippocampus.

When these frequency bands are synchronized in a manner of optimizing connections for specific activities and networks, the brain operates most efficiently.

When a specific frequency is synchronized in the brain, multiple neural networks harmonize and efficiently transmit information.

Brainwave entrainment is a method of regulating rhythms among brain regions using specific frequencies of sound, light, or electrical stimulation. Binaural beats or neurofeedback may be used to induce alpha or gamma waves.

Phase synchronization is a phenomenon in which different brain regions match phases at the same frequency. A representative example is synchronization of theta waves in the hippocampus and frontal lobe when recalling memories.

Cross-frequency coupling (CFC) is a method in which one frequency modulates the cycle of another frequency. A representative example is that gamma waves (i.e., a high frequency wave) modulate the cycle of theta waves (i.e., a low frequency wave) to enhance learning and memory.

This synchronization process optimizes cooperation of neural networks and plays a role in enhancing specific cognitive functions.

Representatively, in relation to enhancement of cognitive functions and memory improvement, activation of gamma waves (30 to 100 Hz) enhances connectivity between the hippocampus and frontal lobe to improve long-term memory and learning ability. Gamma wave synchronization is related to memory recovery in patients with Alzheimer’s disease.

Synchronization of theta waves (4 to 8 Hz ) may enhance creativity and improve problem-solving ability. When theta waves in the hippocampus and frontal lobe are synchronized, creative thinking is activated.

In addition, regarding improvement of attention and executive function, synchronization of beta waves (14 to 30 Hz) may increase concentration and improve working memory. When the DLPFC and occipital lobe are synchronized by beta waves, attention is improved. Research results have shown that neurofeedback through beta wave training is effective for patients with ADHD.

Activation of the SN may regulate switching between the DMN and the CEN to enhance attention and concentration. When the ACC and an anterior insular cortex (aINS) are synchronized, smooth switching from the DMN to the CEN is achieved.

In addition, regarding emotion regulation and mental health improvement, activation of alpha waves (8 to 14 Hz) may regulate the balance between the amygdala and the frontal lobe to relieve stress. When alpha waves increase, anxiety is reduced and a relaxed state is promoted.

Synchronization of gamma waves may promote positive emotions and help prevent depression. When gamma waves are synchronized in the frontal lobe and parietal lobe, happiness increases and cognitive functions are improved.

As will be described later, various methods may be used to induce synchronized oscillations in the brain.

Representatively, tACS may synchronize brain networks by applying a specific frequency. Gamma wave (e.g., 40 Hz) stimulation is effective for improving memory and treating Alzheimer's disease.

Binaural beats are a method of inducing specific brainwaves by playing sounds of different frequencies to both ears. A gamma (e.g., 40 Hz) binaural beat is helpful for improving concentration and memory.

Meditation and breathing training may increase alpha and theta waves to reduce stress and promote creative thinking.

Neurofeedback is a technique of training to control specific brainwaves through real-time brainwave monitoring. It is used for treatment of ADHD, anxiety disorders, and depression.

Photobiomodulation may induce gamma waves using light of a specific frequency (e.g., 40 Hz flickering light).

In conclusion, synchronized oscillations in the brain (e.g., entrainment) play a role in enhancing cognitive functions such as memory, concentration, and emotion regulation by optimizing cooperation among brain regions.

Synchronization of gamma waves (e.g., 40 Hz) strengthens memory and learning, and synchronization of theta waves (4 to 8 Hz) promotes creative thinking.

Methods for inducing synchronized oscillations in the brain may include tACS, binaural beats, meditation, and neurofeedback.

By utilizing these principles, cognitive functions may be optimized and mental health may be improved.

In some embodiments, the synchronized oscillations to be applied may be effectively set in a gamma frequency band of 30 Hz or higher.

The gamma frequency is associated with high-level cognitive functions, attention control, and memory enhancement, and may be used to further enhance activation of the SN and switching to the CEN.

Meanwhile, when the first content is provided in addition to the described gamma frequency stimulation, a noise stimulus that deactivates the SN may be further provided.

The noise stimulus may be provided in a manner of entraining synchronized oscillations associated with SN deactivation and may include an alpha wave (α) of 8 to 13 Hz or a theta wave (θ) of 4 to 7 Hz.

The alpha wave and theta wave are associated with activation of the DMN, and thus may be used to more precisely regulate switching of the brain network.

In some embodiments, the stimulation method may be implemented using transcranial neuromodulation techniques.

These may include at least one of tACS, tDCS, tMS, TUS, tPBM, and sensory stimulation.

The sensory stimulation may be provided in various forms such as visual, auditory, tactile, vibratory, olfactory, and gustatory stimuli, and may optimize the responsiveness of the brain network.

Particularly, as a technical feature proposed in some embodiments, the above-defined stimulation methods may be developed into a multi-stimulation method.

That is, at least two or more of the stimulations such as tACS, tDCS, tMS, TUS, and tPBM may be simultaneously applied, and thereby a stronger effect of network switching may be induced.

In addition, in some embodiments, the first content is noise content, and the second content is mission content related to the user’s goal.

That is, the brain responsiveness may be controlled by selectively providing content that activates the SN.

In some embodiments, the first content may include random sound, and the second content may include at least one of music, environment sound, or voice related to the user.

In some embodiments, a method may be applied in which the second content is learning sound information (e.g., English words and idioms (or phrases)).

In some embodiments, a method may be applied in which the first content is a random image and the second content is composed of an image related to the user.

In some embodiments, a method may be applied in which the first content and the second content simultaneously include sound information and image information, respectively.

In some embodiments, a method may be applied in which the first content is lecture content and the second content is composed of a region including specific important learning content.

In some embodiments, the first content may include random information for guiding movement that does not require concentration, and the second content may include information for guiding movement that requires concentration.

The above specific embodiment will be described with reference to the drawings.

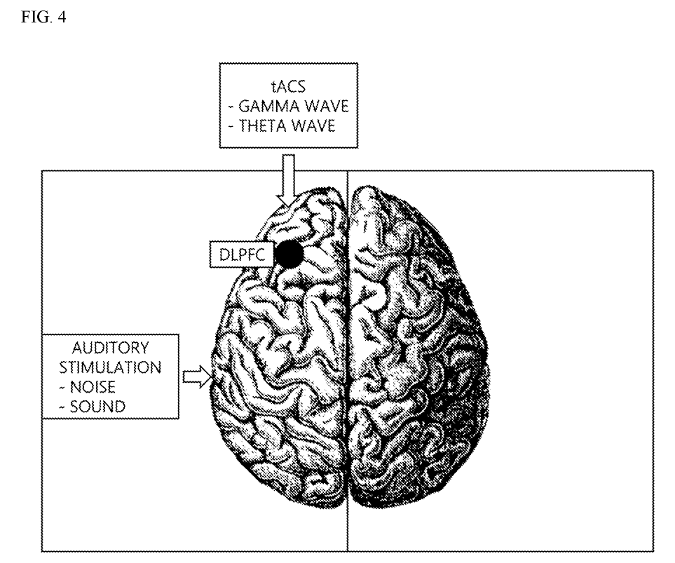

FIG. 4 illustrates an example of network normalization through auditory stimulation.

Referring to FIG. 4, the auditory stimulation is composed of useful sound and useless sound (e.g., noise), and a process of distinguishing them is performed in the brain. The useless sound refers to unnecessary environmental noise or background noise that does not require attention, and the useful sound refers to sound including information such as music, environment sound, and voice. When processing such auditory information, the DLPFC plays an important role, and the information is processed in association with specific brainwave patterns.

The tACS performs a role of determining auditory stimulation information and thereby regulates the activation of brain networks.

A gamma wave (30 to 100 Hz) is activated when the auditory stimulation is determined to be the useful sound, and plays a role in activating the CEN. When the gamma wave increases, attention concentration and information processing centered on the prefrontal cortex are enhanced, and the process of efficiently recognizing important auditory information is facilitated.

A theta wave (4 to 8 Hz) is activated when the auditory stimulation is determined to be the useless sound and plays a role in activating the DMN. When the theta wave increases, unnecessary external stimuli are excluded and internal thought processes are activated, and unnecessary information such as noise is filtered out.

That is, when a specific frequency of the DLPFC is adjusted using tACS, the gamma wave may be increased to process the useful sound and enhance concentration, and the theta wave may be increased to facilitate the filtering process of unnecessary noise. Through this mechanism, the processing of auditory information is optimized, and effective auditory cognitive functions may be performed.

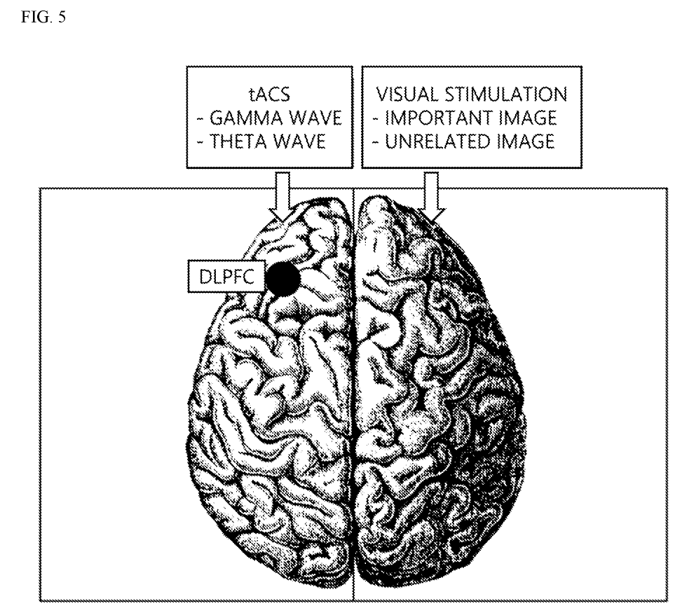

FIG. 5 illustrates an example of network normalization through visual stimulation.

Referring to FIG. 5, the visual stimulation is composed of an important image and unrelated image, and a process of distinguishing them is performed in the brain. The important image refers to visual information that is highly relevant to the user, and the unrelated image refers to visual information that is semantically less related to the user. When processing such visual information, the DLPFC plays a central role, and the information is selected in association with specific brainwave patterns.

The tACS performs a role of determining visual stimulation information and thereby regulates the activation of brain networks.

A gamma wave (30 to 100 Hz) is activated when the visual stimulation is determined to be the important image, and plays a role in activating the CEN. When the gamma wave increases, attention concentration and information processing centered on the prefrontal cortex are enhanced, and the process of efficiently recognizing important visual information is facilitated.

A theta wave (4 to 8 Hz) is activated when the visual stimulation is determined to be the unrelated image, and plays a role in activating the DMN. When the theta wave increases, unnecessary external visual information is excluded and internal thought processes are activated, and attention to unrelated images is reduced.

That is, when a specific frequency of the DLPFC is adjusted using tACS, the gamma wave may be increased to select important images and enhance concentration, and the theta wave may be increased to facilitate the process of filtering unnecessary visual information. Through this mechanism, the processing of visual information is optimized, and effective visual cognitive functions may be performed.

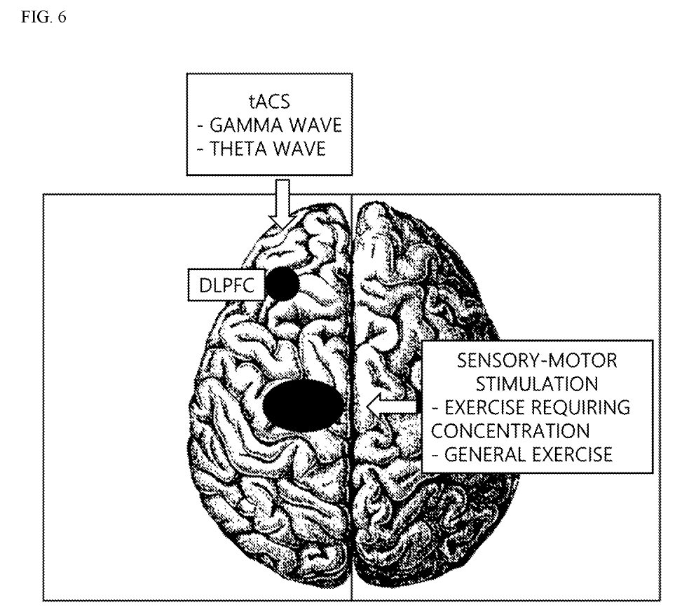

FIG. 6 illustrates an example of network normalization through sensory-motor stimulation.

Referring to FIG. 6, the sensory-motor stimulation is composed of exercises that require concentration and general exercises, and a process of distinguishing them is performed in the brain. The exercises that require concentration are those that demand attention and precise movement, and the general exercises are those that can be performed repetitively without attention. When processing such exercise information, the DLPFC plays a central role and determines the importance of the exercise in association with specific brainwave patterns.

The tACS performs a role of determining motor stimulation information and thereby regulates the activation of brain networks.

A gamma wave (30 to 100 Hz) is activated when the sensory-motor stimulation is determined to be the exercise that requires concentration, and plays a role in activating the CEN. When the gamma wave increases, attention and motor control abilities centered on the prefrontal cortex are enhanced, and a response to exercises that require precise performance and cognitive engagement is facilitated.

A theta wave (4 to 8 Hz) is activated when the sensory-motor stimulation is determined to be the general exercise, and plays a role in activating the DMN. When the theta wave increases, external motor stimuli are filtered and automated motor processes are activated, forming a suitable state for performing repetitive exercises that require little neurological involvement.

That is, by adjusting a specific frequency of the DLPFC using tACS, the gamma wave may be increased to improve the performance of exercises that require precision and concentration, and the theta wave may be increased to optimize the process of performing simple and automated exercises. Through this mechanism, the performance of exercise and the processing of sensory-motor information may be performed efficiently.

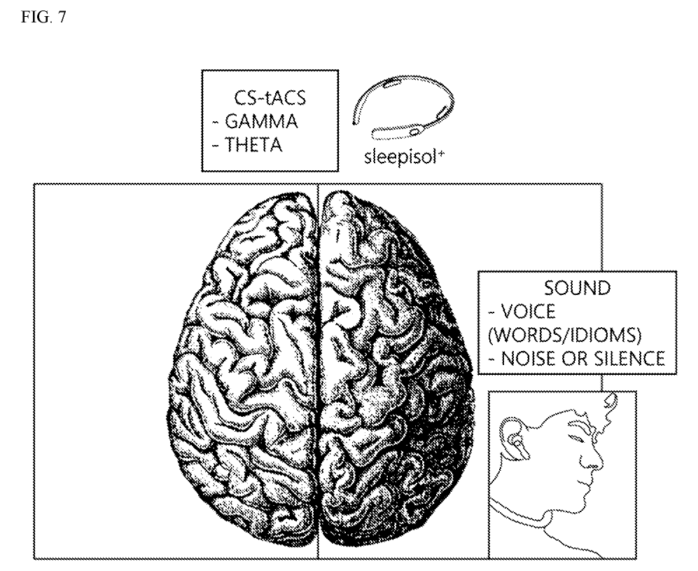

FIG. 7 illustrates an example of network normalization through visual, sound, and tACS stimulation in relation to English learning.

Referring to FIG. 7, a CS-tACS-based English learning assistance system is a technology designed to effectively support memorization of English words and idioms. It operates in a manner that enhances the learner’s selective attention and concentration by combining an auditory stimulation (e.g., sound) system and a transcranial alternating current stimulation (CS-tACS). The auditory stimulation is composed of voice (e.g., words/idioms) and noise (e.g., noise or silence), where the voice stimulation consists of essential English words and idioms for middle and high school students, including content to be memorized by the learner, and the noise stimulation consists of ambient noise or silence, acting as sound not directly related to learning.

The CS-tACS system operates in a manner that optimizes learning effects by synchronizing different brainwaves (e.g., entrainment) depending on the type of auditory stimulation. When the voice is output, gamma entrainment (e.g., gamma wave induction) is activated to stimulate the CEN, thereby enhancing attention and memory; when noise is output, theta entrainment is activated to stimulate the DMN, thereby blocking unnecessary external stimuli and improving internal information processing capabilities.

A system, based on the triple-network model, adjusts the balance among the CEN, DMN, and the SN to optimize the selective attention and concentration necessary for memorizing words and idioms. While the theta entrainment is activated, it suppresses unnecessary noise so that a stable concentration state can be maintained even in noisy or distracting environments, and while the gamma entrainment is activated, it promotes the storage of words and idioms to be learned in the working memory and facilitates their conversion to long-term memory.

Therefore, the CS-tACS-based learning assistance system plays a role in improving concentration and memory by providing an optimal frequency according to the learner’s brain state and supports effective learning of English words and idioms while minimizing the influence of external environments.

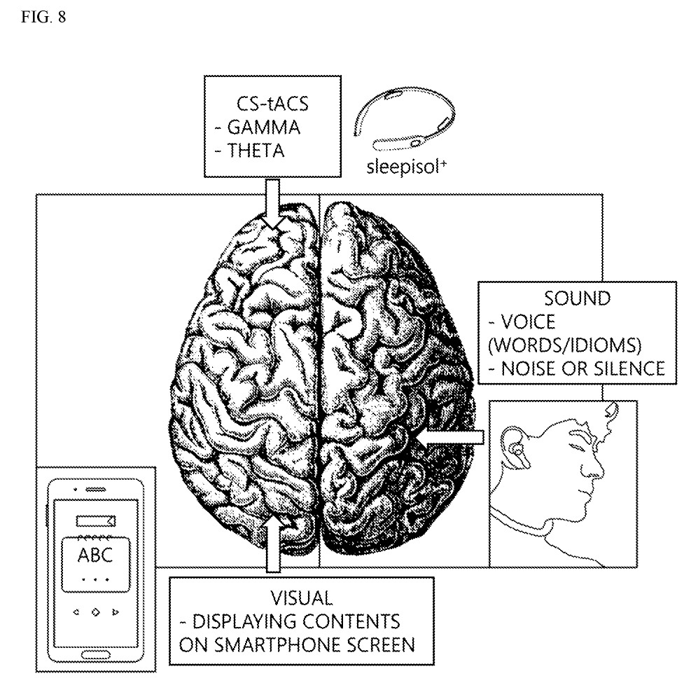

FIG. 8 illustrates an example of network normalization through visual, sound, and tACS stimulation in relation to an internet lecture learning concept.

Referring to FIG. 8, a CS-tACS-based English learning assistance system is a technology designed to more effectively support the memorization of English words and idioms. It operates in a manner that enhances the learner’s selective attention and concentration by combining at least one of auditory stimulation (e.g., sound), visual stimulation (e.g., visual input), and CS-tACS.

The auditory stimulation is composed of voice (e.g., words and/or idioms) and noise (e.g., noise and/or silence), where the voice stimulation is composed of essential English words and idioms for middle and high school students and includes content to be memorized by the learner, and the noise stimulation consists of ambient noise or silence, acting as sound not directly related to learning. In some embodiments, the CS-tACS system may operate in a manner that optimizes learning effectiveness by synchronizing different brainwaves (e.g., entrainment) depending on the type of auditory stimulation. When the voice is output, gamma entrainment is activated to stimulate the CEN, thereby enhancing attention and memory; when the noise is output, theta entrainment is activated to stimulate the DMN, thereby blocking unnecessary external stimuli and improving internal information processing capabilities.

The visual stimulation is performed through a smartphone screen, where the words and idioms to be memorized by the learner are displayed during the memorization process, and unnecessary information is displayed in the standby state to control visual attention switching. These visual elements are combined with auditory stimulation to form a multimodal learning environment, and by simultaneously activating the brain's visual and language processing regions, the learning effect is maximized.

A system, based on the triple-network model, may adjust the balance among the CEN, the DMN, and the SN to optimize the selective attention and concentration necessary for memorizing words and idioms. While the theta entrainment is activated, it suppresses unnecessary noise so that a stable concentration state can be maintained even in noisy or distracting learning environments. While the gamma entrainment is activated, it promotes the storage of the words and idioms to be learned in the working memory and facilitates their conversion to long-term memory.

Therefore, the CS-tACS-based learning assistance system plays a role in supporting the effective learning of English words and idioms by providing an optimal frequency tailored to the learner’s brain state, thereby enhancing concentration and memorization, while minimizing the influence of external environments.

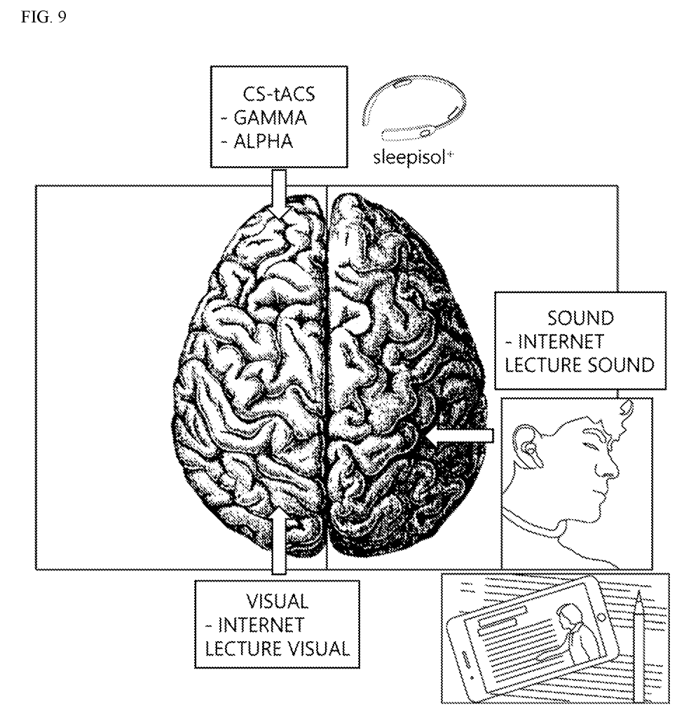

FIG. 9 illustrates an embodiment of utilizing visual, sound, and tACS in relation to an internet lecture learning concept according to an embodiment.

Referring to FIG. 9, the present disclosure is designed to maximize the effectiveness of online lecture learning and is configured to enable a learner to more efficiently remember and utilize lecture content. To this end, it operates by providing optimal neural stimulation according to the learner’s cognitive state through a combination of auditory stimulation (e.g., sound), visual stimulation (e.g., visual input), and transcranial alternating current stimulation (CS-tACS).

The auditory stimulation comprises general sound from the online lecture and plays an important role in enabling the learner to listen to the instructor’s explanation and understand the content. The visual stimulation comprises general visual elements of the lecture video and assists the learner in supplementally learning the lecture content through visual materials. The CS-tACS technology is combined therewith, and appropriate neural stimulation is applied according to the importance of the learning content by using a specific brainwave synchronization (entrainment) technique.

The CS-tACS system may operate based on gamma entrainment (gamma wave induction) and alpha entrainment (alpha wave induction), and provide appropriate brainwave stimulation according to the importance of the content while the learner listens to the lecture. When core content appears, the gamma entrainment (gamma wave induction) is activated, thereby activating the CEN and maximizing concentration and memory. Since the gamma wave play an important role in higher-level cognitive functions and memory formation, stimulating the learner's brain with the gamma wave while learning key concepts enables more effective information retention. On the other hand, when general explanations or supplementary information not related to the core content is delivered, the alpha entrainment (gamma wave induction) is activated, thereby appropriately activating the DMN and enabling the learner to receive information in a more stable state.

The CS-tACS-based online lecture learning system operating in this manner induces gamma waves whenever core content appears in the lecture to maximize the learner’s memory effect, and induces alpha waves when general content is delivered to maintain learning continuity. As a result, the learner does not simply listen to the lecture content, but obtains and stores information in a neuroscientifically optimized manner, thereby improving the overall learning effectiveness.

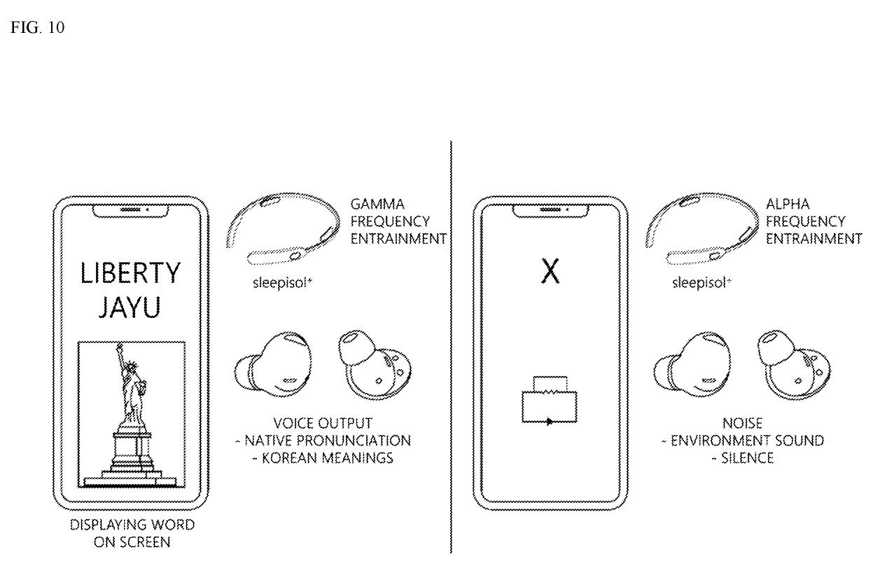

FIG. 10 illustrates an example of implementation of internet lecture learning according to an embodiment.

Referring to FIG. 10, a CS-tACS-based English learning support system may operate in a manner of creating an optimal learning environment according to a learner's cognitive state and automatically distinguish between situations requiring memorization and situations that do not, to induce brainwave synchronization appropriate thereto. When the learner needs to memorize words and idioms (or phrases), gamma frequency entrainment is activated, thereby activating the CEN and promoting memory storage of the learning content. In this process, the smartphone screen displays the words and idioms to be memorized, related images are provided to assist associative learning, and voice output including native pronunciation and Korean meanings is activated, thereby enabling effective learning using both visual and auditory modalities.

Conversely, when the learner is in a state not requiring memorization, theta frequency entrainment (e.g., theta wave induction) is activated, and the DMN is stimulated so that the brain may naturally rest in a state unrelated to learning. In this case, the smartphone screen does not display the words to be memorized or outputs unnecessary information to reduce visual stimulation, and ambient environment sound or silence is provided as auditory stimulation to suppress external stimuli, thereby helping the learner maintain a stable state without distraction.

As a result, the system may induce gamma or theta waves depending on the learner's cognitive state, thereby increasing concentration when necessary so that words and idioms can be effectively memorized, and minimizing external stimuli when learning is not required to enable natural consolidation of information. Through this, the learner may continuously perform efficient learning without being affected by the surrounding environment and may maximize long-term learning effects through systematic attention control.

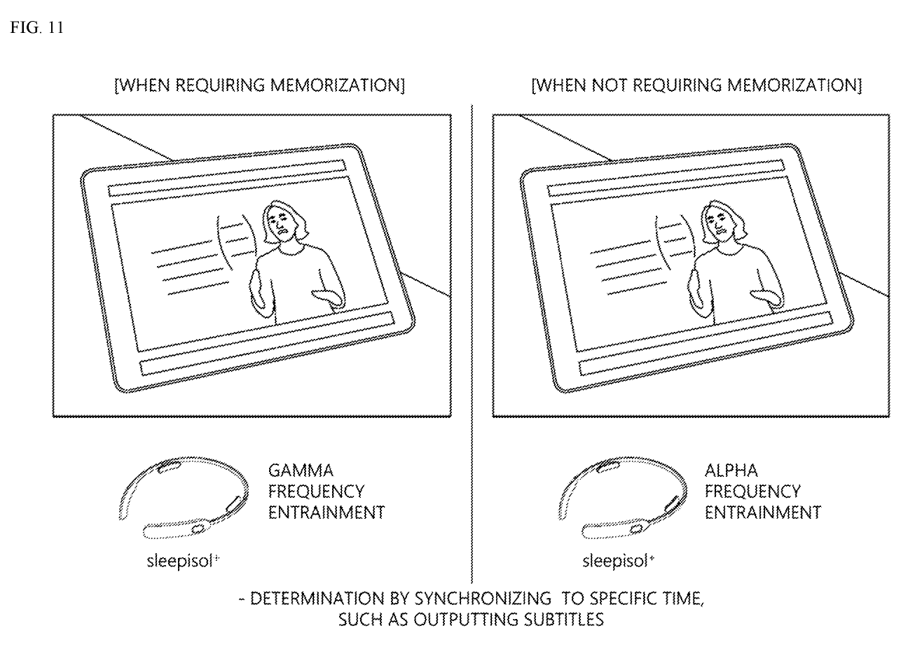

FIG. 11 illustrates an embodiment applied to internet lectures.

Referring to FIG. 11, a CS-tACS-based learning support system operates by synchronizing optimal brainwaves according to a learner's cognitive state in a video-based learning environment to maximize concentration and learning efficiency. When the learner is in a state requiring memorization, gamma frequency entrainment (e.g., gamma wave induction) is activated, thereby stimulating the CEN and improving concentration and memory regarding the learning content. In this process, subtitles of the video content are activated to help the learner acquire key concepts more effectively.

Conversely, when the learner is in a state not requiring memorization, alpha frequency entrainment (e.g., alpha wave induction) is activated, and the DMN is activated to alleviate tension in the learner and to allow natural consolidation of the learning content. In this case, the subtitles are minimized or removed so that the learner may reduce the burden of information acquisition while still maintaining the learning flow.

The system automatically switches the learning mode by synchronizing with the timing of the subtitles in the video content, thereby operating in a manner of maximizing the learner’s concentration when necessary and naturally easing it when not. As a result, the learner may continue efficient learning while minimizing cognitive burden and may maximize long-term learning effects through optimized attention control.

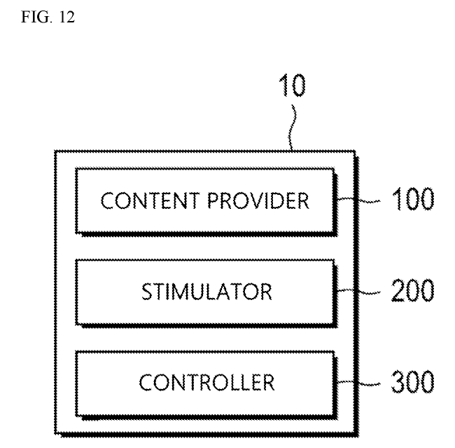

Meanwhile, FIG. 12 illustrates an example of a brain network stimulating apparatus and system according to an embodiment.

Referring to FIG. 12, a system 10 may include a content provider 100 configured to distinguish between preset first content and preset second content and provide them to a user, a stimulator 200 configured to deliver a preset stimulus to the user’s brain, and a controller 300 configured to deliver the stimulus to the user’s brain only when the second content is provided to the user, thereby activating the SN and inducing the brain to transition from the DMN to the CEN.

The specific technologies and embodiments described with reference to FIGS. 1 through 11 can all be applied in a converted form to the apparatus and system shown in FIG. 12.

Meanwhile, a method according to an embodiment may be applied to patients with disorders.

For example, in case of a patient with depression, it may be possible to suppress overactivation of the DMN through SN activation and improve the function of the CEN, thereby alleviating depressive symptoms.

In case of a patient with an anxiety disorder, a therapeutic effect may be expected by normalizing overactivation of the SN and balancing between the DMN and the CEN.

In case of a patient with schizophrenia, symptoms such as delusions and hallucinations may be alleviated by controlling the functions of the SN and the CEN.

In case of a patient with ADHD, it may be possible to improve attention and inhibition control functions by enhancing the function of the CEN.

Meanwhile, the content of the present disclosure may also be applied to the treatment of degenerative brain diseases other than the aforementioned mental disorders.

Ultimately, the present disclosure may balance the brain network based on the triple-network model and assist in the treatment of mental disorders and the improvement of cognitive functions.

According to an embodiment related to depression, based on the triple-network model, it is possible to analyze the functional connectivity of brain networks in patients with major depressive disorder (MDD) and bipolar disorder (BD) to identify commonalities and differences between the two disorders.

Existing studies have analyzed the brain networks of patients with depression from the perspective of static functional connectivity (FC), but it is possible to consider temporal changes in the brain network by measuring dynamic functional network connectivity (dFNC).

The study subjects consisted of 51 BD-II patients, 51 MDD patients, and 52 healthy controls who did not take medication. Brain network data were collected from all subjects using resting-state functional magnetic resonance imaging (RS-fMRI). Researchers analyzed the dFNC using independent component analysis (ICA), sliding window correlation techniques, and k-means clustering, and compared differences among the BD, MDD patients, and the healthy control group.

The triple-network model comprises the CEN, DMN, and SN, and abnormal interactions among these networks are closely related to mental illnesses such as depression. The DMN is a network associated with self-reflection and is overactivated in patients with depression, leading to increased negative thinking and rumination. The CEN is a network responsible for attention and working memory, and its function is impaired in patients with depression, resulting in reduced cognitive ability. The SN is a network that regulates switching between the CEN and the DMN, and in patients with depression, this function is weakened, making emotional regulation and attention switching difficult.

The results of the study showed that patients with BD and MDD had decreased variability in dFNC between the DMN and the CEN compared to healthy controls. In particular, MDD patients showed a distinct decrease in dFNC variability between the anterior DMN (aDMN) and the right CEN (rCEN), whereas BD patients showed a decrease in dFNC variability between the posterior DMN (pDMN) and the right CEN (rCEN). This indicates that both BD and MDD patients have impaired information processing and decision-making abilities, and especially in MDD patients, functions related to self-reflection are more severely damaged.

In this study, the dynamic changes in brain networks were classified into four states and analyzed. States 1, 2, and 4, which show dense functional connectivity, reflect normal brain network functions, while State 3, which shows sparse functional connectivity, reflects a state of impaired information processing. The study found that patients with BD and MDD spent more time in State 3 and had lower frequencies of switching to normal states compared to healthy controls. This suggests that patients with depression and bipolar disorder have difficulty returning to a normal information processing state and that this reduced flexibility of brain networks is a key neurobiological characteristic of mental disorders.

The results of this study imply that balancing the triple-network model by activating the CEN through gamma stimulation and suppressing the DMN through theta or alpha stimulation may be effective in treating depression, and that the treatment method using CS-tACS based on this may have high potential to promote the recovery of neural function in patients with depression.

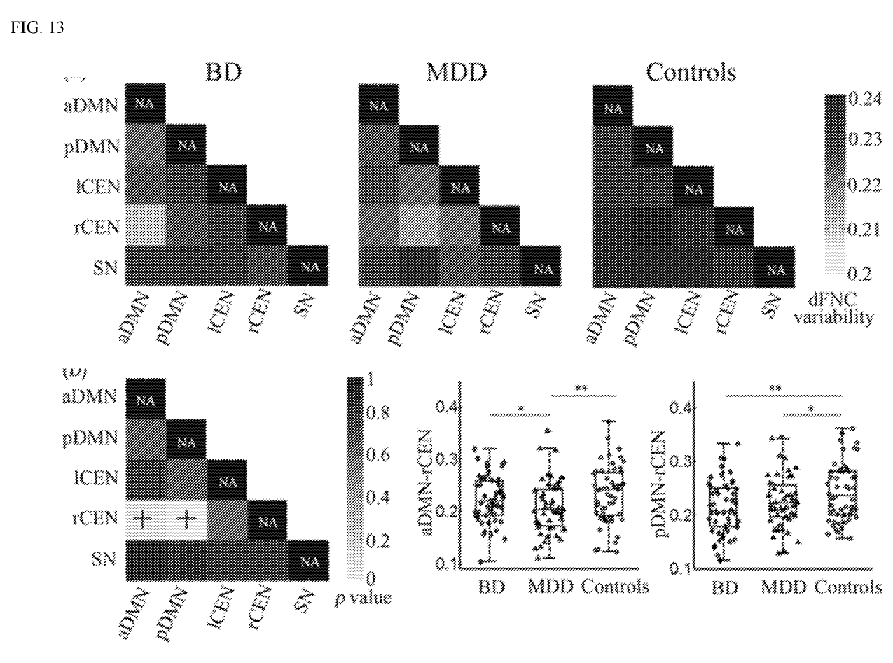

FIG. 13 is a diagram illustrating a graph of dFNC variability analysis and a chart comparing connectivity between networks.

Referring to FIG. 13, it shows a graph visualizing changes in dFNC in the triple-network model and a chart comparing connectivity between networks. A first image illustrates that the dFNC variability between the pDMN and the rCEN in patients with BD and patients with MDD has decreased. Additionally, in patients with MDD, the dFNC variability between the aDMN and the rCEN is further reduced, indicating that functions related to self-reflection are more impaired.

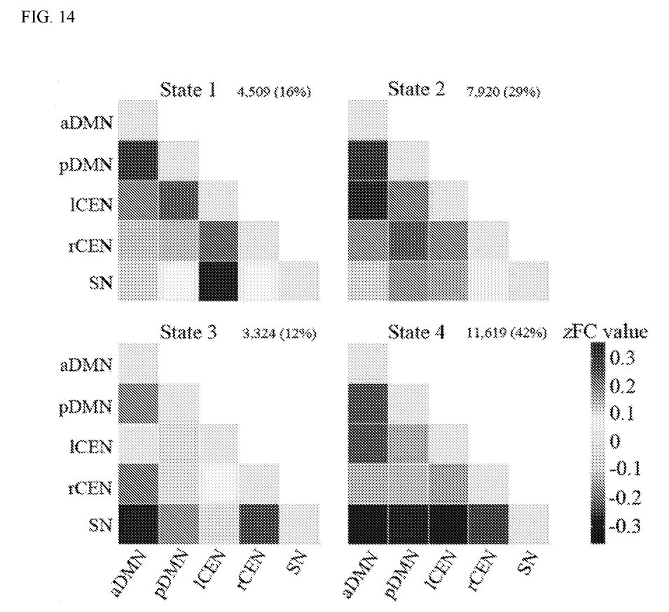

FIG. 14 is a chart illustrating a dFNC state analysis.

Referring to FIG. 14, patients with BD and MDD spent more time in a state (State 3) characterized by sparse functional connectivity compared to normal states (State 1, 2, 4), and showed reduced ability to transition to a normal state.

These results suggest that a neural modulation technique based on the triple-network model may be a promising method for treating mental disorders. In particular, it neurobiologically demonstrates that activating the CEN through gamma entrainment and suppressing the DMN through theta or alpha entrainment may be effective for treating depression.

These results increase the likelihood that non-invasive brain stimulation techniques such as CS-tACS may be practically applied to the treatment of depression and suggest that adjusting the imbalance of the triple-network model may improve the fundamental pathology of mental disorders such as depression.

In some embodiments, a brain network stimulating method, apparatus, and system may activate the SN of the brain through a specific stimulus upon the provision of preset content, and induce a transition from the DMN to the CEN.

The present disclosure may provide an effect of facilitating a functional transition of the brain network. By distinguishing between preset first content and second content and providing them separately, it is possible to adjust the delivery of the preset stimulus to the user's brain under specific conditions only. The stimulus activates the SN of the brain, and the activated SN induces the transition from the DMN to the CEN, thereby improving the user's attention and cognitive function.

In addition, the present disclosure may provide effects of improving working memory and cognitive flexibility. When a specific stimulus is delivered to the DLPFC, the SN is activated, thereby reinforcing the process in which the DMN is suppressed and the CEN is activated. As a result, the user may enhance working memory and cognitive flexibility and more efficiently store information and adapt to new information.

In addition, the present disclosure may provide effects of enhancing attention control and inhibition control. By promoting SN activation and the transition to the CEN, the user's attention control and inhibition control functions are enhanced. This allows the user to concentrate on specific information even in a distracting environment and increases the ability to suppress unnecessary actions, thereby enabling effective response even in situations that require a high level of concentration.

In addition, the present disclosure may provide an effect of activating neural networks through multi-sensory stimulation. The present disclosure enables the application of multi-modal stimulation using at least one of tACS, tDCS, tMS, TUS, tPBM, and sensory stimulation. When these various stimuli are applied in combination, they may achieve greater effects than a single stimulation method and may enable the activation of neural networks utilizing multiple senses such as vision, hearing, and touch.

In addition, some embodiments may provide an effect of activating the CEN by utilizing high-frequency gamma oscillation. By applying synchronized oscillation (e.g., entrainment) and using gamma oscillation of 30 Hz or higher to activate the SN, it is possible to enhance the user's concentration and promote the activation of the CEN. Such gamma oscillation plays an important role in learning and improving cognitive functions, and more precise control may be achieved by designing it to be entrained only when specific mission content is provided.

In addition, some embodiments may provide an effect of blocking unnecessary information and optimizing brain function. By designing the user's brain to distinguish between content with low relevance (e.g., random sound, random image, etc.) and content with high relevance (e.g., music, environment sound, voice, etc.), meaningless stimuli (e.g., noise) may be induced to activate the DMN, and meaningful stimuli may be induced to activate the CEN. In addition, by using alpha waves (α) at 8 to 13 Hz or theta waves (θ) at 4 to 7 Hz, neural responses to unnecessary information may be regulated, thereby inducing selective responses to information requiring concentration.

In addition, some embodiments may provide an effect of improving neural network imbalance in patients with mental disorders. The present disclosure may be utilized for treating various mental disorders by training the transition between the DMN and the CEN through activation of the SN. In case of patients with depression, it may alleviate excessive activation of the DMN and functional decline of the CEN. In case of patients with anxiety disorders, it may help solve the problem of hyperactivation of the SN and imbalance between the DMN and the CEN. In addition, in case of patients with schizophrenia, it may improve the functional decline of the CEN, and in case of patients with ADHD, it may supplement attention deficit by activating the CEN.

In addition, some embodiments may provide an effect of improving learning and motor performance ability. By associating specific mission content with learning sound (e.g., English words and idioms (or phrases)) or designating key learning content of lecture content as a specific region and providing it accordingly, the learner's memory and comprehension may be improved. In addition, by distinguishing between exercise requiring concentration and exercise not requiring concentration and applying specific stimuli accordingly, motor performance ability may be enhanced. This may be applied not only in learning environments but also in sports and rehabilitation therapy.

The recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise.

Although the above-described embodiments have been described as utilizing aspects of the presently invented subject matter in one or more stand-alone computer systems, the disclosure is not limited thereto and may also be implemented in conjunction with any computing environment such as networks or distributed computing environments. Furthermore, aspects of the subject matter of the disclosure may be implemented across multiple processing chips or devices, and storage may be similarly affected across multiple devices. Such devices may include PCs, network servers, and portable devices.

While the disclosure has been described in connection with certain embodiments, various modifications and alterations may be made without departing from the scope of the invention as understood by those skilled in the art to which the invention pertains. Such modifications and alterations should be regarded as falling within the scope of the claims appended hereto.

The effects provided in the disclosure are not limited to those described above but may be extended to various aspects that can be derived from some embodiments described below.

Meanwhile, although the disclosure has been described in connection with certain embodiments, various modifications and changes may be made without departing from the scope of the invention as understood by those skilled in the art to which the invention pertains. Such modifications and changes should be regarded as falling within the scope of the claims appended to the disclosure.

What is claimed is:

1. An apparatus comprising:
a content provider configured to provide preset first content and preset second content to a user in a respective manner;
a stimulator configured to deliver a preset stimulus to a brain of the user; and
a controller configured to control the stimulator such that the stimulus is delivered to the brain of the user only when the second content is provided to the user,
wherein the stimulus is delivered to the brain of the user only when the second content is provided to the user, thereby activating a Salience Network (SN) and inducing a transition of the brain from a Default Mode Network (DMN) to a Central Executive Network (CEN).

2. The apparatus of claim 1, wherein the stimulus is delivered to a Dorsolateral Prefrontal Cortex (DLPFC) region to activate the SN.

3. The apparatus of claim 2, wherein, when the SN is activated and the brain transitions from the DMN to the CEN,
at least one function among working memory, cognitive flexibility, executive function, decision making and problem-solving, attention control, and inhibition control is performed.

4. The apparatus of claim 2, wherein the stimulus is configured to activate the SN by entraining synchronized oscillations in a plurality of regions of the brain.

5. The apparatus of claim 4, wherein the entrained synchronized oscillations comprise gamma oscillations of 30 Hz or higher.

6. The apparatus of claim 5, wherein, when the first content is provided to the user,
the stimulator is further configured to deliver a preset noise stimulus to the brain of the user,
wherein the noise stimulus is a stimulus entraining synchronized oscillations associated with deactivation of the SN, and
wherein the synchronized oscillations entrained by the noise stimulus comprise alpha waves (α) of 8 to 13 Hz or theta waves (θ) of 4 to 7 Hz.

7. The apparatus of claim 5, wherein the stimulus comprises at least one of:
transcranial alternating current stimulation (tACS),
transcranial direct current stimulation (tDCS),
transcranial magnetic stimulation (tMS),
transcranial ultrasound stimulation (TUS),
transcranial photobiomodulation (tPBM), and
sensory stimulation, and
wherein the sensory stimulation comprises at least one of:
visual stimulation, auditory stimulation, tactile stimulation, olfactory stimulation, and gustatory stimulation.

8. The apparatus of claim 7, wherein the stimulus for the entrainment comprises multi-stimulation in which at least two or more among the tACS, tDCS, tMS, TUS, tPBM, and sensory stimulation are simultaneously applied.

9. The apparatus of claim 7, wherein the first content is noise content having a low degree of relevance to the user, and
wherein the second content is mission content having a high degree of relevance to the user.

10. The apparatus of claim 9, wherein the first content comprises sound information including at least a random sound,
wherein the second content comprises sound information including at least one of music related to the user, environment sound related to the user, and voice related to the user, and
wherein only when the second content is provided, the gamma oscillation is entrained, and the gamma oscillation entrainment activates the SN to induce a transition from the DMN to the CEN.

11. The apparatus of claim 10, wherein the second content comprises learning sound information including at least one of preset English words and preset idioms that the user intends to learn.

12. The apparatus of claim 9, wherein the first content comprises image information including at least a random image unrelated to the user,
wherein the second content comprises image information including at least an image related to the user, and
wherein only when the second content is provided, gamma oscillation is entrained, and the gamma oscillation entrainment activates the SN to induce a transition from the DMN to the CEN.

13. The apparatus of claim 9, wherein the first content comprises both sound information including at least a random sound, and image information including at least a random image unrelated to the user,
wherein the second content comprises both sound information including at least one of music related to the user, environment sound related to the user, and voice related to the user, and image information including at least an image related to the user, and
wherein only when the second content is provided, gamma oscillation is entrained, and the gamma oscillation entrainment activates the SN to induce a transition from the DMN to the CEN.

14. The apparatus of claim 9, wherein the first content is lecture content information that the user is learning,
wherein the second content is specific lecture region information including preset important learning content among the lecture content information, and
wherein only when the second content is provided, gamma oscillation is entrained, and the gamma oscillation entrainment activates the SN to induce a transition from the DMN to the CEN.

15. The apparatus of claim 9, wherein the first content is random information guiding the user's movement performed without concentration by the user, and
wherein the second content is information guiding the user's movement that requires concentration by the user, and
wherein only when the second content is provided, gamma oscillation is entrained, and the gamma oscillation entrainment activates the SN to induce a transition from the DMN to the CEN.

16. The apparatus of claim 7, wherein when the user is a patient with depression, the stimulus activates the SN, and based on the activation of the SN, the transition process between the DMN and the CEN is trained, thereby solving a problem of excessive activation of the DMN and functional degradation of the CEN in the patient with depression.

17. The apparatus of claim 7, wherein when the user is a patient with an anxiety disorder, the stimulus activates the SN, and based on the activation of the SN, the transition process between the DMN and the CEN is trained, thereby solving a problem of excessive activation of the SN and imbalance between the DMN and the CEN in the patient with an anxiety disorder.

18. The apparatus of claim 7, wherein when the user is a patient with schizophrenia, the stimulus activates the SN, and based on the activation of the SN, the transition process between the DMN and the CEN is trained, thereby solving problems of functional degradation of the CEN and abnormality of the SN in the patient with schizophrenia.

19. The apparatus of claim 7, wherein when the user is a patient with ADHD, the stimulus activates the SN, and based on the activation of the SN, the transition process between the DMN and the CEN is trained, thereby solving a problem of functional degradation of the CEN in the patient with ADHD.

20. A brain network monitoring method comprising:
providing preset first content and preset second content to a user in a respective manner;
delivering a preset stimulus to a brain of the user;
controlling so that the stimulus is delivered to the brain of the user only when the second content is provided to the user; and
monitoring whether the brain transitions from a Default Mode Network (DMN) to a Central Executive Network (CEN) by activating a Salience Network (SN) through the stimulus.
